(19) United States
(12) Patent Application Publication
(10) Pub. No.: US 2010/0044228 A1
(43) Pub. Date: Feb. 25, 2010

(54) Multi-Dimensional Analysis

(76) Inventors: Marc Baumann, Helsinki (FI); Samuli Franssila, Helsinki (FI); Zuzana Demianova, Helsinki (FI); Eemeli Pöysä, Espoo (FI); Risto Kostiainen, Helsinki (FI); Tapio Kotiaho, Espoo (FI)

Correspondence Address: RYAN, MASON & LEWIS, LLP, 90 FOREST AVENUE, LOCUST VALLEY, NY, 11560, US

(21) Appl. No.: 12/302,439

(22) PCT Filed: May 22, 2007
(86) PCT No.: PCT/IB2007/002448

§ 371 Date: Sep. 30, 2009

(30) Foreign Application Priority Data
May 26, 2006 (GB) 0610555.5

(51) Int. Cl. G01N 33/559; G01N 27/00

(52) U.S. Cl. 204/456; 204/606; 204/610

(57) ABSTRACT

A device and a method for multidimensional separation and analysis of molecules is disclosed. The device comprises a chamber for subjecting a first substance to a first analysis step and a space for receiving a second substance. The device is configured to apply pressure to the second substance to move the second substance towards a product of the first analysis step for providing a sample for a second analysis step.

### FIELD OF THE INVENTION

The present invention relates to an analysis device and method. More particularly the present invention relates to a multi-dimensional biomolecular analysis and separation and to an analysis device configured to perform such an analysis and separation. The analysis may comprise at least in part electrophoresis. Furthermore, certain embodiments relate to two- or multi-dimensional electrophoresis separation.

### BACKGROUND OF THE INVENTION

A two-dimensional gel electrophoresis (2-DE) is a method for separating complex mixtures of proteins and other biomolecules into two analyses. It has been found to be a useful technique for separating complex mixtures of proteins that is in most instances more efficient in providing a higher resolving power than what is obtainable by means of one-dimensional separations.

O'Farrell, P. H., 1975, J. Biol Chem. 250, 4007-4021, describes an example of a classical method comprising two electrophoresis steps, isoelectric focusing (IEF) and sodium dodecylsulphate polyacrylamide gel electrophoresis (SDS PAGE), carried out separately and successively. Electrophoresis using 2-DE on a polyacrylamide gel (PAGE) can separate proteins in a sample according to isoelectric point (pI) and molecular weight. This method is often referred to as 2-D PAGE. The classical 2-D PAGE generally provides isoelectric focusing electrophoresis in one direction (first dimension) followed by polyacrylamide gel electrophoresis in a second direction (second dimension). The 2-D PAGE can be useful in analyzing protein composition of a sample solution, such as a biological sample. For example, gene activity may be studied by analyzing various proteins important to certain cellular functions. Examples of candidate sample compounds may further comprise, but are not limited to, drugs, proteins, nucleic acids, peptides, metabolites, biopolymers and other simple or complex substances.

In addition to the classically known two-dimensional gel electrophoresis, one can also combine other separation parameters to be understood as two-dimensional gel electrophoresis. For example, one can separate proteins under acidic conditions and then transfer these separated samples to the second dimension which might be a SDS-PAGE. Furthermore, the first dimension can include any type of separation which is suitable for the analytes of interest. Also the second dimension can be of any physical or chemical constellation instead of an SDS-PAGE. This could include a tris-tricine gel electrophoresis, especially suitable for small molecular weight analytes.

The isoelectric focusing electrophoresis (IEF) is typically performed in polyacrylamide gel strips or gel rods with pH gradients. Immobilized pH gradients fixed within the gel may be used. In an alternative, ampholytes are applied together with a sample. Ampholytes and molecules comprised in the sample are subjected to an electric field. The ampholytes migrate into their specific isoelectric region and establish and stabilize the pH gradient. Each molecule species of the sample migrate in the electric field to a position where the gradient has a pH value corresponding to the isoelectric point of the molecule species. Molecule species, such as proteins, are thus separated in the first dimension based on isoelectric properties of the molecule species. The gel comprising the molecule species separated in the first dimension may be subjected to appropriate treatments, such as mobilization or washing, if desired.

The second dimension, SDS PAGE, is typically performed in another electrophoresis device on an SDS PAGE gel. The gel strips or rods treated in the first dimension are placed on one end of the SDS PAGE gel. An electric field applied to the gels causes the molecule species separated in the first dimension to be transferred to the SDS PAGE gel. In the SDS PAGE gel, the molecule species are separated by electrophoresis based on molecular mass of each molecule species.

Despite its good resolving power, 2-D PAGE has not been generally adopted for high throughput screening studies. This is because the first and second dimension gels are typically run separately. Two different running devices are thus typically required which makes the automation thereof troublesome. In addition, a high level of operator skill and knowledge may be required to successfully complete a 2-D gel.

In a typical scheme removal of as much of the operator intervention as possible should assist in the aim of achieving a truly high throughput and reproducibility of 2-D gels. A way to accomplish this would be to combine the first and second dimension gels in a single analysis device and thus remove the need for the operator to interface the gels manually. Combining the two gels in a single device should also improve possibilities for automation. However, even if the gels were placed together in a single device the two steps of the analysis should still be separated. This may require keeping the gels separated until the transfer of molecules from the first to the second dimension is required. However, it may be technically very difficult to keep the gels separated in a single apparatus without a need to relocate at least one of the gels after the first analysis step.

U.S. Pat. Nos. 4,483,885 and 4,874,490 each propose use of removable isolation between the gels, thus enabling the two separations in succession without the need to manually insert or remove a gel. More particularly, U.S. Pat. No. 4,483,885 discloses a “spacer film” that can be removed between the analysis steps to bring the gels into contact with each other. U.S. Pat. No. 4,874,490 discloses an “insulating layer” that can be removed to allow the gel to come into contact with each other.

Patent application US 2004/0144647 A1 also discloses an electrophoresis device for a two-dimensional electrophoretic separation of macromolecules. The device of US 2004/0144647 A1 has a first separation channel for performing isoelectric focusing. The device of US 2004/0144647 A1 has capillary channels orthogonal to the first separation channel for performing separation according to molecular mass. Between the first separation channel and the capillary channels there may be a porous or permeable partition plate and a gap. By applying a high voltage, analyte molecules may be transported from the first separation channel through the partition wall to the gap for concentrating. The concentrated analyte molecules may be dosed into the capillary channels by applying a further voltage.

However, it might be desired to be able to perform the analysis without a need for capillary separation, or without a need for removal of an isolation member between the substances. It might also be desired to avoid using further chemical substances in the process.

Furthermore, miniaturizing or reducing a scale of the analysis device may be desired. Examples of miniaturized one- and two-dimensional electrophoresis devices have also been published in various articles, for example Sluszny, C. and Yeung, E. S., 2004, Analytical Chemistry, Vol. 76, No. 5, 1359-1365; Neuhoff, V., 2000, Electrophoresis, 21, 3-11; Chen, X. et al., 2002, Anal. Chem., 74, 1772-1778; Berdichevsky, M., Khandurina, J. and Guttmann, A., 2003, American Biotechnology Laboratory, January 2003, 22-23. The motivation for changing from large-scale analysis instrumentation to micro-scale may have several advantages, for example sample consumption may be reduced, analysis times may be faster, high-throughput analysis may become possible. Furthermore, single-use devices may suppress cross-contamination.

### SUMMARY OF THE INVENTION

The present invention aims to address at least some of the issues mentioned above, and to provide a novel way of providing the multi-dimensional processes in a device. In an embodiment, a small-scale device is provided for separating a set of predefined samples for e.g. clinical diagnosis. In an embodiment, the device may also be used for an analysis of a subset of samples at predefined conditions, such as narrowed pH range.

In accordance with an aspect of the invention, there is provided a device for multidimensional separation and analysis of molecules. The device comprises a chamber for subjecting a first substance to a first analysis step and a space for receiving a second substance. The device is configured to apply pressure to the second substance to move the second substance towards a product of the first analysis step for providing a sample. The sample may then be used in a second analysis step.

In accordance with another aspect of the invention, there is provided a method for multidimensional separation and analysis of molecules. The method comprises performing a first analysis step on a first substance and applying pressure to a second substance for moving the second substance towards the product of the first analysis step to receive a sample. A second analysis step is then performed on the sample.

In a more detailed embodiment a stopper membrane is provided for separating the first substance and the second substance. The stopper membrane may be provided with a first surface facing the first substance and a second surface facing the second substance, the device being configured to press the second substance towards the stopper membrane. The thickness and material of the stopper membrane is preferably selected such that the stopper membrane allows movement of the product of the first analysis step there through whilst preventing undesired movement of the first and second substances. An electric field may be provided for drawing the product of the first analysis step into contact with the second substance through the stopper membrane. According to an embodiment diffusion is utilized in drawing the product of the first analysis step into contact with the second substance.

The device may be configured to apply pressure to the second substance by reducing the volume of a channel containing said second substance. The device may be configured to apply mechanical pressure to the second substance. According to a possibility gas-compression and/or hydraulic pressure is used. The second substance may be expanded by means of adding a further substance thereto. Pressure may also be applied to the first substance.

The device may be configured for two-dimensional gel electrophoresis. The first substance may comprise a first dimension gel and the second substance may comprise a second dimension gel. At least one of the first analysis step and the second analysis step may comprise independently at least one of separating, cleaning, decontaminating and recovering chemical substances.

The analysis device may be provided in a microchip.

Various other aspects and embodiments of the invention shall become clear from the following description and figures.

### BRIEF DESCRIPTION OF FIGURES

The invention will now be described in further detail, by way of example only, with reference to the following examples and accompanying drawings, in which:

FIGS. 1 to 3 show an analysis device according to an embodiment;

FIGS. 4 to 6 show an analysis device according to another embodiment; and

FIG. 7 shows a flow chart according to an embodiment.

### DETAILED DESCRIPTION OF EMBODIMENTS

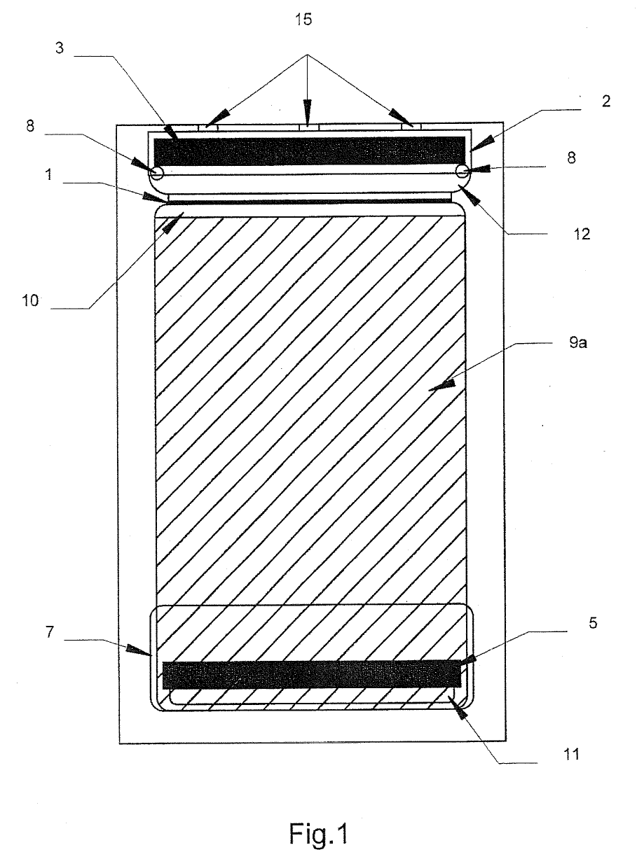

FIGS. 1 to 3 are views of an automated two-dimensional gel electrophoresis device in accordance with an embodiment. The device comprises a first cavity or volume or similar chamber 12 for receiving a first substance for first analysis phase, such as separation. The first substance may comprise a first dimension gel. The device further comprises a second cavity or volume 10 for receiving a second substance 9 for a second analysis phase, the second analysis phase using at least partially a product of the first analysis phase. The second substance may comprise a second dimension gel. In the configuration shown in FIGS. 1 to 3 the first chamber can also be termed upper chamber and the second chamber can be termed a lower chamber.

The first dimension gel in chamber 12 can be for example in a rod or strip format. An example is a 2×1×24 mm strip of gel. It is noted that this is only a non-limiting example that is mentioned to give an idea of the magnitude of the possible size of the first dimension gel. The first dimension gel provides an isoelectric focusing strip in its own cavity. The second dimension gel 9 may be provided in the second cavity in a slab format.

Both gels may be supplied from respective buffer reservoirs. The device of FIGS. 1 to 3 is shown to comprise a first buffer chamber 2 for the first dimension gel and a second buffer chamber 7 for the second dimension gel.

Buffer channels or conduits 15 are provided for loading analysis substances in the first chamber 12. The first chamber 12 is configured to perform the IEF of a two-dimensional electrophoresis. An IEF electrode 8 and IEF gel 12 are shown to be provided in the upper chamber. In an alternative, other electrophoresis may be performed in the first chamber, examples comprising, but not limiting to, electrophoresis using a native gel, an acid gel, a tris-trisine gel suitable for e.g. small molecules. Further examples of possible analysis steps to be carried out in the first chamber may comprise any other separation method suitable for separating molecules, in particular biomolecules, based on, for example, physical or chemical characteristics of said molecules. Further non-limiting examples of possible analysis steps to be carried out in the first chamber comprise cleaning, decontaminating and recovering chemical substances, and so on.

A first electrode 3 is located in the first chamber for applying a voltage in the first chamber. A second electrode 5 is provided in the second chamber 7 for applying a voltage in the second chamber through buffer reservoirs. It is noted that the electrodes can be arranged in any appropriate manner in the chambers. A buffer channel 6 is provided for supplying the buffer chamber 7 with further substance.

In an embodiment the focusing gel is focused in the first chamber after which procedure the second dimension gel is pushed towards a space or volume 10 that is provided in close proximity to the first chamber. The space 10 may be provided by a gap, channel or any other conduit or volume that can be located between the first dimension gel and the second dimension gel and can receive the second gel. The space 10 is preferably provided underneath the first gel 12. The space is preferably almost as long as the first gel 12.

The pushing of the second dimension gel 9 towards the first gel is effected by applying pressure thereto. The pressure may be applied until the second gel reaches the end of the space 10 and the lower surface of a stopper membrane 1. After the second gel has reached the stopper membrane, the pressure can either be removed or be kept constant so as to maintain the position of the second gel.

The stopper membrane 1 provided between the first chamber 2 and the space 10 is preferably non-removable. The stopper membrane 1 acts as a stopper for the second dimension gel 9 when it is pressed to enter the space 10. The first dimension gel is preferably in contact with the upper surface of the membrane. The two gels 12 and 9 are thus separated by the membrane 1.

The dimensions and material or materials of the stopper membrane 1 are preferably selected such that it does not prevent the movement of the samples there through, but functions for preventing the downwards movement of the first gel and upwards movement of the pressurised second gel. Thus the samples i.e. products of the first dimension step can move downwards through the stopper membrane 1 from the first gel to the second gel, for example after an appropriate electrical field has been switched on. The gels itself are separated by a distance that equals with the thickness of the membrane. The thickness of the stopper membrane is preferably selected to be in the range of a few micrometers to a few hundred micrometers.

In certain embodiments it may be useful to pressurise also the first gel. The pressure may be provided to maintain the position of the first gel and to ensure good contact between the first gel and the first electrodes.

The gels can be pressurised by various means. For example, gas-compression, hydraulic pressure or mechanical force can be used to pressurize a gel. The gel can also be expanded with various liquids, for example by adding water to the gel through the lower buffer reservoir to make the gel to swell.

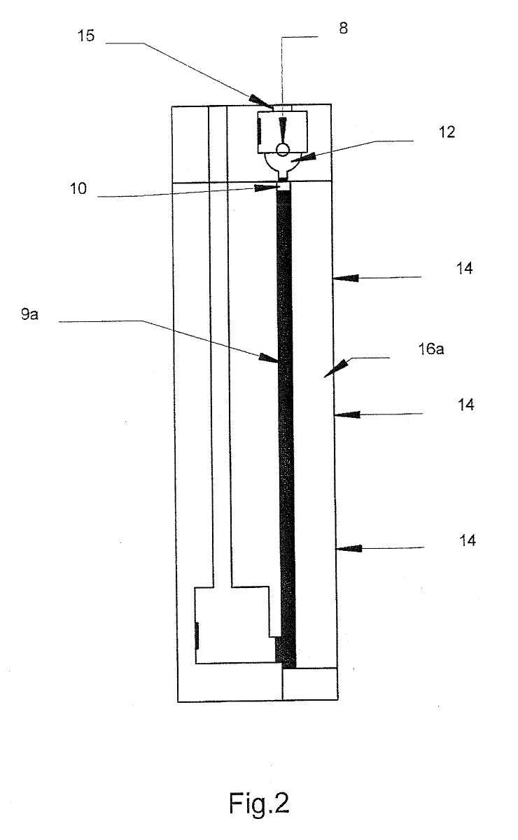

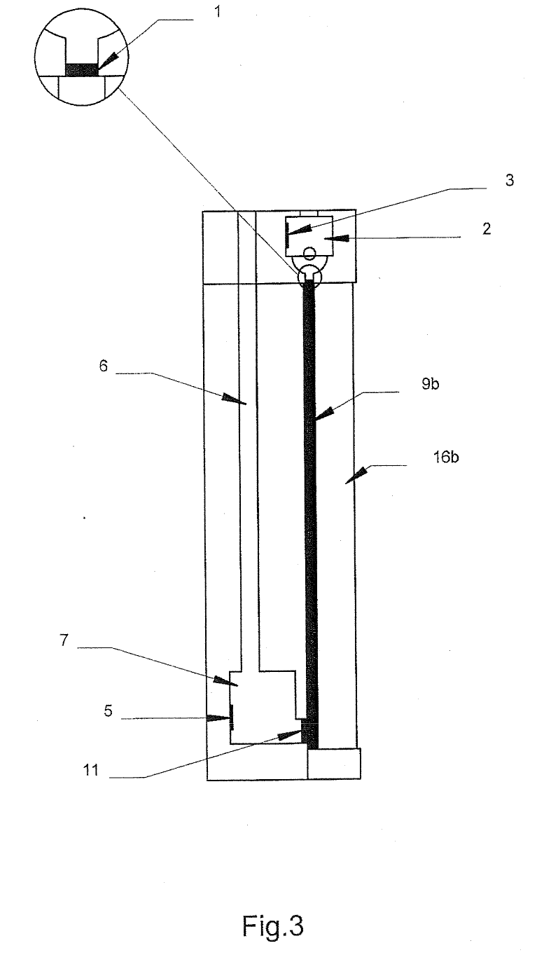

In FIGS. 1 to 3 the second gel is pressurised, the pressure being provided by a pressurizing unit 16. Arrows 14 in FIG. 2 illustrate how pressure can be applied to the second gel by the pressurising unit 16. The pressurising unit 16 is in a non-pressurised state in FIG. 2. After application of pressure (arrows 14) to the back of the unit 16, see FIG. 3, the unit 16 moves towards the slab gel 9, thus applying pressure to the gel.

The device of the embodiment of FIGS. 1 to 3 is manufactured from a number of components. This may be advantageous, for example, for the purposes of testing prototypes, optimizing the dimension and/or materials used, and for manufacturing purposes, especially if only a few devices of a kind are manufactured.

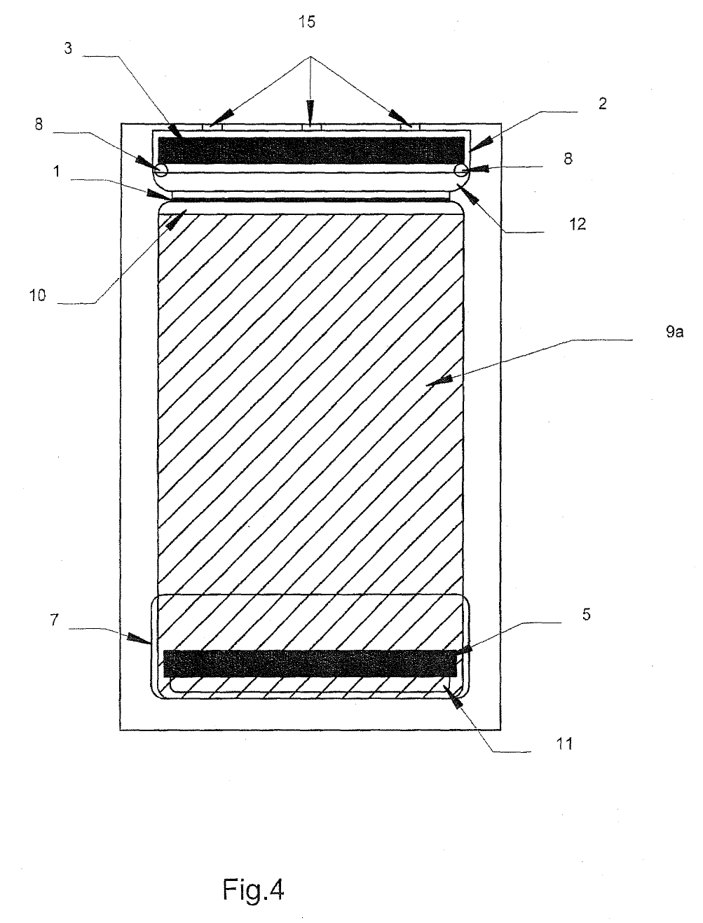

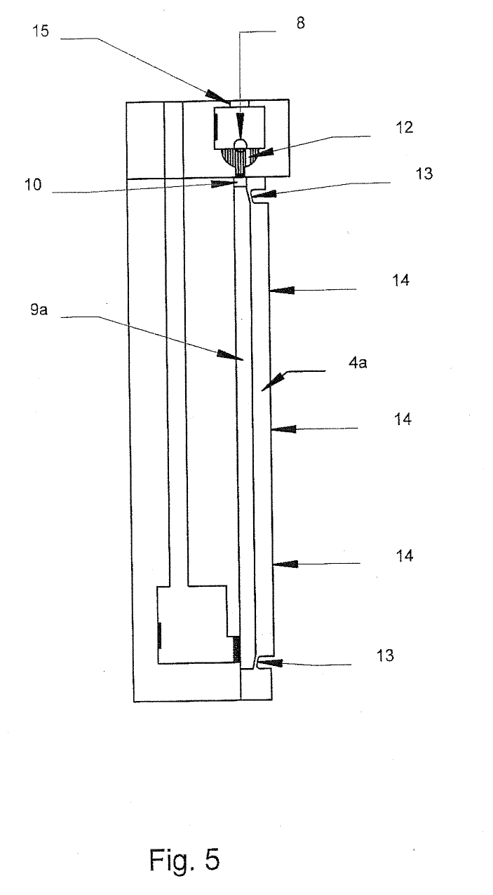

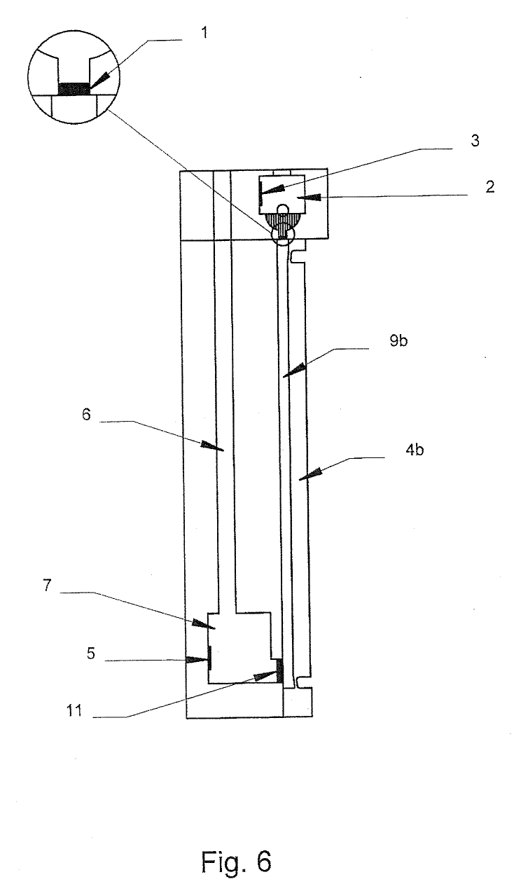

A device may also be provided that comprises only a few components, or only one component as will be explained below referring to FIGS. 4 to 6. Therefore, it shall be appreciated that, even if FIGS. 1 to 3 show the device having separate components, the invention is not limited to this embodiment. One or more of the components may also be changed in different steps. Furthermore, the device may comprise additional components, some examples of which shall be mentioned in the following.

FIGS. 4 to 6 show a device where a pressurising unit 4 is provided such that it forms an integrated part of the device body. The unit is attached to the body by flexible joints 13 such that application of pressure 14 to one side of the unit 4 causes it to lessen the volume in a channel 9, thus pressing the gel in the channel towards a space 10 below a membrane 1.

Although not shown in the enclosed Figures, the device may comprise further analysis units configured to perform further analysis steps of a multi-dimensional analysis. Further analysis units may be positioned above and/or under the first chamber. For example, a third analysis unit positioned above the first chamber may be configured to decontaminate a sample. For example, a fourth analysis unit may be configured to react with a product from the analysis. This reaction may be, but is not limited to, an antibody binding, an enzyme reaction, or the like. Free selection of a type and a number of analysis and units may allow performing a wide range of different analysis and separations in a device.

The different parts of the device may be of any appropriate material. The material should be compatible, preferably inert, with analysis substances and samples, such as with PAA and proteins. Appropriate materials may comprise, but are not limited to, poly(dimethylsiloxane) (PDMS), polycarbonate (PC), polymethyl metacrylate (PMMA), glass or any glass-like material and silicon. Channels, wells, cavities, and other compartments of the device may be etched, embossed, machined or otherwise produced in the parts of the device.

The isolation membranes may be of any appropriate material. The material should be compatible, preferably inert, with analysis substances and samples, such as with PAA and proteins. Appropriate materials may comprise, but are not limited to, polyvinylidene chlorine (PVDC), poly(dimethylsiloxane) (PDMS), polycarbonate (PC), polyvinylidene difluoride (PVDF), cellulose, nitrocellulose, polymethyl metacrylate (PMMA), nylon, or any other acrylic polymer or polycarbonate or Teflon, or other polymer. A requirement for the membrane material is that it mechanically prevents excessive and unwanted movement of the gels while it allows analytes to penetrate there through.

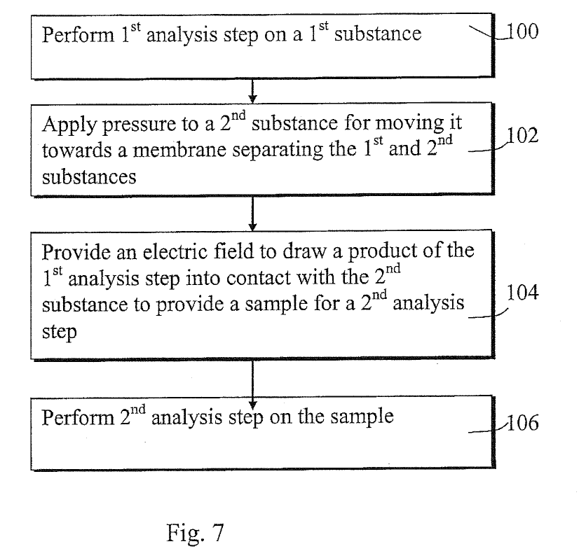

FIG. 7 shows a flow chart illustrating a method according to an embodiment of the invention. In step 100, a first analysis step is performed on a first analysis chamber. The first analysis step is for separating a composition from a first substance to produce a sample. In step 102, a pressure is applied to a second gel so as to push it towards a stopper membrane isolating the second gel from entering the first chamber. The purpose of this step is to move by pressure the second gel into proximity with the first substance and/or the sample produced by the first step. The pressure is preferably increased until the gel contacts the stopper membrane. In step 104, an electric field is provided to draw the sample through the stopper membrane and into contact with the second gel. In step 106, a second analysis step is then performed on a further sample provided by the product of the first analysis step and the second substance.

According to an embodiment diffusion is utilized in drawing the product of the first analysis step into contact with the second substance. This is based on the realization that if the first substance and the second substance are held in contact to each other, diffusion will cause the analytes to move downwards, thus providing the sample for the second analysis step. Diffusion may be used alone, or in combination with an electric field for drawing the product of the first step through the membrane.

In an embodiment, a device for gel electrophoresis is provided in a microchip. The microchip may be provided with at least one channel or conduit where a substance such as a gel can be moved by means of applying pressure thereto.

Embodiments of the invention provide a device capable of running two or more analysis steps, such as a full two-dimensional gel electrophoresis or another multi-dimensional biomolecular analysis, in a single analysis device. Implementation in a single device may allow avoiding laborious workflow of known 2-DE devices. In a particular embodiment a miniaturized device is provided. The device may be provided from a single work-piece or cast, or from substantially low number of component.

In addition to the examples above, the first gel may comprise a gel which is run under acidic conditions or a gel which has at least one additional component included. The additional component may be provided, for example, by antibodies or selected antigenes, enzyme substrates or epitope recognition motifs. These may be covalently or non-covalently bound to the gel matrix. The second gel may also comprise a native (i.e. non-SDS) gel or any gel which is run under conditions where the analyte does not loose its native fold or structure. Tris-Tricine gel or a simple agarose that is suitable for separation of e.g. DNA and RNA molecules may also be used.

Although the invention has been described in the context of particular embodiments, various modifications are possible without departing from the scope and spirit of the invention as defined by the appended claims. It should be appreciated that whilst experimental examples illustrating the present invention have mainly been described in relation to two-dimensional electrophoresis, embodiments of the present invention may be applicable to other types of multi-dimensional electrophoresis or other types of multiple step separations or analyses.

1.-35. (canceled)

36. A device for multidimensional separation and analysis of molecules comprising:
a chamber for subjecting a first substance to a first analysis step;
a space for receiving a second substance comprising a second dimension gel;
wherein the device is configured to apply pressure to the second dimension gel to move the second dimension gel towards a product of the first analysis step for providing a sample for a second analysis step.

37. A device according to claim 36, comprising a stopper membrane having a first surface facing the first substance and a second surface facing the second substance, wherein the device is configured to press the second substance towards the stopper membrane.

38. A device according to claim 37 being configured to bring the first substance into contact with the first surface of the stopper membrane and the second substance into contact with the second surface of the stopper membrane.

39. A device according to claim 37, wherein the thickness and material of the stopper membrane is selected such that the stopper membrane allows movement of the product of the first analysis step there through whilst preventing undesired movement of the first and second substances.

40. A device according to claim 36, wherein the device is configured to apply pressure to the second substance by reducing the volume of a space containing said second substance or by means of gas-compression.

41. A device according to claim 36, wherein the device is configured to apply at least one of mechanical pressure and hydraulic pressure to the second substance.

42. A device according to claim 36, wherein the second substance is expanded by means of adding a further substance thereto.

43. A device according to claim 36 being configured for two-dimensional gel electrophoresis.

44. A device according to claim 36, wherein the first substance comprises an isoelectric focusing (IEF) gel.

45. A device according to claim 36, wherein the first gel comprises one of a gel which is run under acidic conditions and a gel which has at least one additional component included.

46. A device according to claim 36, wherein the second gel comprises a sodium dodecylsulphate (SDS) gel.

47. A device according to claim 36, wherein the second gel comprises a gel that can be run under conditions where the product of the first analysis step does not loose its native fold or structure.

48. A device according to claim 36, comprising a pressurizing member that is attached to the body of the device by flexible joints such that application of pressure to one side of the pressurizing member causes it to lessen the volume in a conduit containing the second substance.

49. A method for multidimensional separation and analysis of molecules, the method comprising:
performing a first analysis step on a first substance;
applying pressure to a second substance comprising a second dimension gel for moving the second dimension gel towards a product of the first analysis step to receive a sample; and
performing a second analysis step on the sample.

50. A method according to claim 49, wherein the step of applying pressure comprises pressing the second substance towards a stopper membrane located between the first substance and second substance.

51. A method according to claim 50, comprising providing electric field for drawing the product of the first analysis step into contact with the second substance through the stopper membrane.

52. A method according to claim 49, wherein the step of applying pressure comprises at least one of reducing the volume of a channel containing said second substance, applying mechanical pressure to the second substance, subjecting the second substance to gas-compression, applying hydraulic pressure to the second substance, and expanding the second substance by adding a further substance thereto.

53. A method according to claim 49, comprising the further step of wherein the step of applying pressure to the first substance.

54. A method according to claim 49, comprising two-dimensional gel electrophoresis.

55. A microchip for gel electrophoresis comprising
a chamber for subjecting a first substance to a first analysis step; and
at least one conduit for a gel, the microchip being configured to apply pressure to the gel in the conduit to press the gel towards a product of the first analysis step for providing a sample for a second analysis step.
